(19) United States
(12) Patent Application Publication
(10) Pub. No.: US 2007/0038126 A1
(43) Pub. Date: Feb. 15, 2007

(54) System and method for monitoring of end organ oxygenation by measurement of in vivo cellular energy status

(76) Inventors: Jason L. Pyle, Redwood City, CA (US); Alexander M. Aravanis, San Francisco, CA (US); Jeffrey N. Roe, San Ramon, CA (US); Myer H. Rosenthal, Palo Alto, CA (US)

Correspondence Address: WILSON SONSINI GOODRICH & ROSATI, 650 PAGE MILL ROAD, PALO ALTO, CA, 94304-1050, US

(21) Appl. No.: 11/473,976

(22) Filed: Jun. 23, 2006

Related U.S. Application Data
(60) Provisional application No. 60/595,337, filed on Jun. 23, 2005.

(51) Int. Cl. A61B 6/00

(52) U.S. Cl. 600/476

(57) ABSTRACT

A method is provided of measuring in vivo of an endogenous fluorophore in a tissue site. A known excitation wavelength of the endogenous flurophore is selected within a range of wavelengths at which the endogenous flurophore undergoes fluorescence. The tissue site is irradiated with irradiated light having at least the selected excitation wavelength within the range of wavelengths. A fluorescence emission of the tissue site resulting from the irradiation thereof is detected. A relative or absolute concentration of the endogenous fluorophore is determined by multiplying it by a calibration factor that depends one at least one of, a known excitation and emission property of the endogenous fluorophore, an intensity of the irradiated light, optical properties of an excitation probe, and specific properties of the tissue. The relative or absolute concentration of the endogenous fluorphore is used to estimate at least one of a, in vivo cellular energy production status or state of end-organ tissue oxygenation.

### CROSS-REFERENCE TO RELATED APPLICATIONS

This application claims the benefit of U.S. Ser. No. 60/595,337, filed Jun. 23, 2005, which application is fully incorporated herein by reference.

### BACKGROUND OF THE INVENTION

1. Field of the Invention

This invention relates generally to systems and methods for measuring relative or absolute concentrations of endogenous flurophores in a tissue site, and more particularly to systems and methods for measuring, in vivo the relative or absolute concentration of endogenous flurophores in a tissue site.

2. Description of the Related Art

Cells produce and metabolize molecules which have intrinsic light emitting properties. Some of these molecules, such as NADH, are intimately related to the biochemical pathway responsible for oxygen metabolism. During glycolysis, a single molecule of glucose is converted into two molecules of glyceraldehyde-3-phosphate (G-3-P). The energy of the subsequent G-3-P oxidation reaction is conserved in the formation of NADH from NAD+. In the presence of oxygen, the conversion of pyruvate to acetyl-CoA yields two molecules of NADH. Three more molecules of NADH are formed for every turn of the citric acid cycle. Thus, the process of glucose metabolism in the presence of oxygen generates a total yield of ten NADH molecules for every molecule of glucose. The ten molecules of NADH are converted into thirty molecules of ATP as electrons pass from NADH to molecular oxygen through a chain of electron carriers. Therefore, the stoichiometry of NADH/NAD+ is shifted heavily toward the production of NAD+ in the presence of oxygen; while, the stoichiometry of NADH/NAD+ is shifted toward NADH in anaerobic conditions. Measurement of cellular NADH reflects a direct measurement of the energy production status of a cell, a process intimately tied to the availability of molecular oxygen.

In the setting of the hospitalized patient, cellular energy production is most frequently compromised by inadequate oxygenation of end-organ tissues. Even when cardiac output and measured oxygen saturation of hemoglobin are normal, end-organ tissues may still not receive adequate oxygenation. This condition is especially worrisome during the inflammatory processes that accompany sepsis and septic shock, as well as in the presence of the many vasoactive substances used in anesthesia and critical care.

There is a need for a system and methods for active monitoring of the intrinsic fluorescent properties of cells to measure their energy production status. There is a further need for a system that allows this measurement to be performed in vivo, such that the oxygenation and energy productions status can be monitored.

### SUMMARY OF THE INVENTION

Accordingly, an object of the present invention is to provide methods and systems that actively monitor the intrinsic fluorescent properties of cells to measure their energy production status.

Another object of the present invention is to provide methods and systems that monitor the intrinsic fluorescent properties of cells to measure their energy production status in vivo, such that the oxygenation and energy productions status can be monitored.

These and other objects of the present invention are achieved in a method of measuring in vivo of an endogenous flurophore in a tissue site. A known excitation wavelength of the endogenous flurophore is selected within a range of wavelengths at which the endogenous flurophore undergoes fluorescence. The tissue site is irradiated with irradiated light having at least the selected excitation wavelength within the range of wavelengths. A fluorescence emission of the tissue site resulting from the irradiation thereof is detected. A relative or absolute concentration of the endogenous flurophore is determined by multiplying it by a calibration factor that depends one at least one of, a known excitation and emission property of the endogenous flurophore, an intensity of the irradiated light, optical properties of an excitation probe, and specific properties of the tissue. The relative or absolute concentration of the endogenous flurophore is used to estimate at least one of a, in vivo cellular energy production status or state of end-organ tissue oxygenation.

In another embodiment of the present invention, a method of measuring in vivo of endogenous flurophores in a tissue site Irradiates the tissue site at multiple irradiation wavelengths with excitation wavelengths known to excite one or more selected endogenous flurophores. A measured emission wavelength response of an emitted light intensity for one or more of the excitation wavelengths is received. An equation is formed for each measured emission wavelength, where the measured emission wavelength is equal to a sum of the responses from the selected endogenous fluorophores. A system of equations is formed where there is an equation for each combination of an irradiation wavelength and measured emission wavelength.

In another embodiment of the present invention, a method is provided for monitoring energy production status of in vivo tissue. Emission of first and second groups of endogenous flurophores are measured. The first group has quantity or emitting characteristics that vary with cellular respiration of oxygen, and the second group has quantity or emitting characteristics that vary with energy production status of mammalian cells. A determination is made of a concentration of one or more of the endogenous fluorophore. A determination is made of a relative or absolute concentration of the endogenous fluorophore by multiplying it by a calibration factor that depends one at least one of, a known excitation and emission property of the endogenous fluorophore, an intensity of the irradiated light, optical properties of an excitation probe, and specific properties of the tissue. The relative or absolute concentration of the endogenous fluorophore is sued to estimate at least one of a, in vivo cellular energy production status or state of end-organ tissue oxygenation.

In another embodiment of the present invention, a system is provided for measuring in vivo at least one endogenous fluorophores in a tissue site. A light source produces an excitation wavelength of the endogenous flurophore within a range of wavelengths at which the endogenous flurophore undergoes fluorescence. A detector detects a fluorescence emission of the tissue site resulting from the irradiation thereof. A processor is provided that analyzes the detected emission to determine the presence of the endogenous flurophore in the tissue site.

### BRIEF DESCRIPTION OF THE DRAWINGS

FIG. 1 is a flow chart of one embodiment of the present invention, illustrating a method and system for measuring, in vivo, the relative or absolute concentration of endogenous fluorophores in a tissue site.

FIG. 2 is a flow chart of another embodiment of the present invention, illustrating a method and system for measuring, in vivo, the relative or absolute concentration of endogenous fluorophores in a tissue site.

FIG. 3 is a schematic diagram of one embodiment of a system of the present invention.

### DETAILED DESCRIPTION

In one embodiment of the present invention, a method is provided for measuring, in vivo the relative or absolute concentration of endogenous fluorophores in a tissue site. Suitable tissue sites include but are not limited to oral mucosa, esophageal mucosa, gastric mucosa, intestinal mucosa, bladder mucosa, and the like.

For the purpose of this specification, the synonymous terms, ‘intrinsic emission molecule’ and ‘endogenous fluorophore’ both refer to a molecule naturally present in mammalian cells that emits light following absorption of electromagnetic energy (fluorescence or phosphorescence) or chemical action (luminescence). In general, the method of monitoring the energy production status of end organ tissues is performed by transmitting excitation light onto the end organ tissue and measuring the fluorescence of intrinsic emission molecules. The close relationship between the fluorescence of intrinsic emission molecules, such as NADH, and the cellular energy production status, has been well established in the scientific literature. By measuring the fluorescence emission of intrinsic emission molecules at the site of end organ tissues, such as the mucosa of the gastrointestinal tract, end organ energy production status can be monitored in a medically beneficial manner. Moreover, this also allows for the medically beneficial monitoring of in vivo tissue oxygenation since this parameter is closely related to the end-organ energy production status.

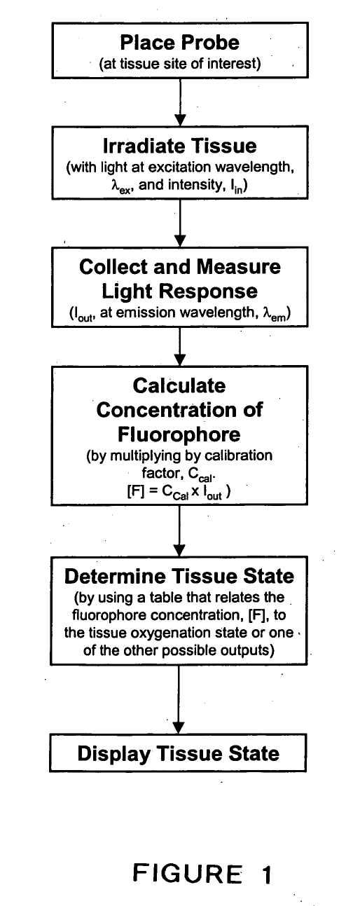

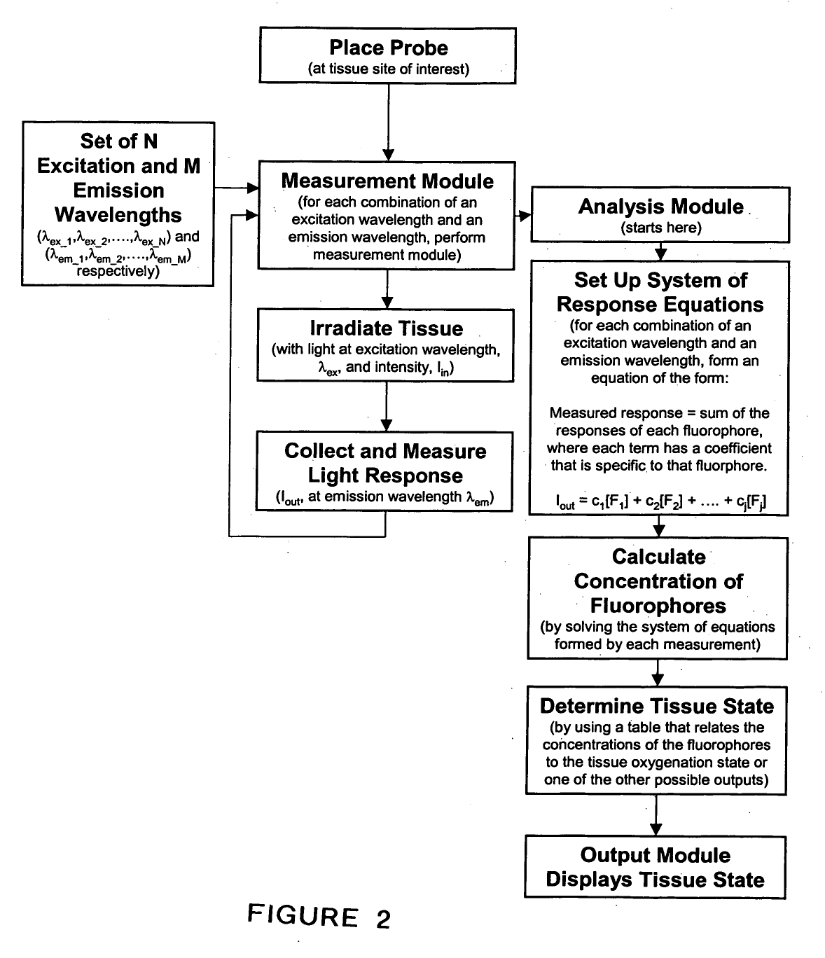

A known excitation wavelength is selected for the endogenous flurophore in a range of wavelengths at which the endogenous flurophore and/or chromophore undergoes fluorescence. The tissue site is irradiated with radiation having at least the selected excitation wavelength within the range. Fluorescence emission is detected of the tissue site resulting from the irradiation thereof. The detected emission is analyzed to determine the relative or absolute concentration of the endogenous flurophore and/or chromophore in the tissue site. The measurements are used to estimate the in vivo cellular energy production status and state of end-organ tissue oxygenation, as illustrated in the flow charts of FIGS. 1 and 2

In one embodiment, following irradiation of the tissue site, the intensity of the emitted light is measured for a wavelength known to be in the emission spectra of this fluorophor. An estimate or calculation is then made of the relative or absolute concentration of the fluorophore or by multiplying the intensity by a calibration factor that depends on but is not limited to one or more of the following: the known excitation and emission properties of the fluorophore, the intensity of the irradiated light, the optical properties of the measurement system, and the specific properties of the tissue site being irradiated. From the estimate or calculation of the concentration of this fluorophore, estimations are then made for the important clinical parameters of in vivo cellular energy production status and state of end-organ tissue oxygenation. This estimate is based either upon known relations between the concentration of this fluorophore and these clinical parameters or upon an experimentally derived calibration. This result is then displayed in one or more forms described hereafter.

In another embodiment, the tissue site is irradiated at multiple wavelengths known to excite one or more fluorophores interest. For each excitation wavelength, the intensity of the emitted light is measured at one or more wavelengths known to be in the emission spectra of one or more fluorophores. For each measurement, involving irradiation at a certain wavelength and a measured response at a certain emission wavelength, an equation is created in the form where the measured response is equal to the sum of the responses from all the fluorophores. The term representing the response of each fluorphore is proportional to the fluorphore concentration and one or more constants, some of which may represent the unique spectral properties of the particular fluorophore. It should be noted that the concentration of certain fluorphores are closely related to tissue oxygenation state whereas others do not vary with tissue oxygenation state and whose emission signals represent background signals.

A system of equations is formed, where there is an equation for each combination of irradiation wavelength and measured emission wavelength. This system of equations is solved for the absolute or relative concentrations of each of the fluorophores. In another embodiment, instead of solving the system of equations directly, the ratios of measurements at different excitation and/or emission wavelengths is performed to obtain the concentration of one or more flurophores of interest. The value of doing measurements at multiple excitation and emission wavelengths is that it allows for correction of background fluorphores, instrumental variation, and positioning of the probe. From the calculation of the concentration of these one or more fluorophores, an estimation is then made of the in vivo cellular energy production status and state of end-organ tissue oxygenation. This estimate is based either upon the known relations between the concentrations of these fluorophores and the in vivo cellular energy production status and state of end-organ tissue oxygenation or upon an experimentally derived calibration. These results are then displayed.

In one embodiment, a system is provided that can be a bedside instrument of electronic and optical components connected to a disposable catheter that is designed for insertion into the end organ of interest. A splanchnic perfusion bed and its close relationship to the blood flow in other important end organs, such as the liver and kidney, is particularly useful for end-organ oxygenation monitoring. Currently used catheters, placed either through the nose or mouth, into the gut represents one embodiment of a method for the placement of an end organ oxygenation monitor. Loss of end-organ oxygenation results in ischemia, infarction, and eventual death of the important visceral organs. The monitoring of the energy status of end organ tissues is also achieved with the methods and systems of the present invention.

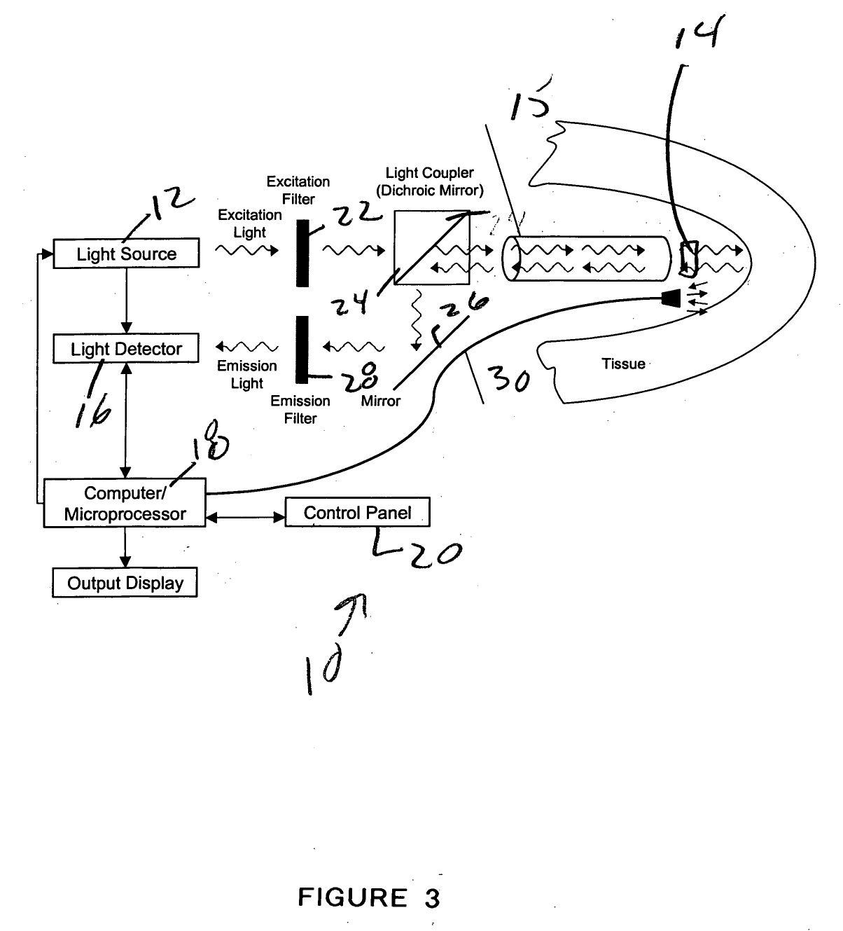

In one embodiment of the present invention, as illustrated in FIG. 3, a system 10 is provided for measuring in vivo the endogenous fluorophores in the tissue site. A light source 12 is provided that produces light at a wavelength in the excitation spectra of one or more of the endogenous flurophores. A probe 14 can be provided. The probe 14 can be coupled to the light source with a coupler such as a light conduit 145, and the like The tissue is irradiated with the excitation light emanating from the probe 14. The same probe 14 then collects the light emanating from the tissue site and transmits it to a detector 16 via the light conduit. The detector 16 measures this light, which is composed of emitted light from the fluorphores and possibly some of the excitation light that was incident on the irradiation site that has been scattered and reflected back into the probe. Suitable detectors include but are not limited to a photodiode, avalanche photodiode, charge-coupled devices (CCDs), photo-multiplier tubes (PMTs) and the like. Some embodiments might require multiple light sources if one source cannot provide all the required excitation wavelengths. Some embodiments might have multiple detectors which would allow simultaneous measurements at different wavelengths, rather than doing them sequentially.

Resources, including but not limited to a processor 18, analyze the detected emission to determine the presence of the endogenous flurophore in the tissue site. The processor 18 has sufficient memory and processing power to control the light source and detector, store the measured values of emission light, calculate the fluorophore concentrations using the analysis algorithm, determine the tissue state based on the fluorophore concentrations, and display this to an output device such as an LED panel, LCD screen, or CRT screen.

For all combinations of specified excitation wavelengths λₑₓ(1,2, . . . Nₑₓ) and emission wavelengths λₑₘ(1,2, . . . Nₑₘ) the processor 18 executes the information as shown in FIGS. 1 and 2.

For each excitation and emission wavelength pair, the sample response is represented as a sum of the responses from each chemical specie, and the processor 18 can execute the following algorithm:

1 D, a constant factor representing illumination intensity and wavelength independent effects on the optical pathway

2. λₑₓ(i), the ith excitation wavelenth

3. λₑₘ(k), the kth emission wavelength

4. Cⱼ, the concentration of the jth chemical species

5. N_{c}, the number of chemical species

6. Tj(λₑₓ(i), λₑₘ(k)), the energy transfer function of the jth chemical species when illuminated at ith excitation wavelength and measured at the kth emission wavelength

7. R(λₑₓ(i), λₑₘ(k)), the measured sample response when it is illuminated at ith excitation wavelength and the response is detected at the kth emission wavelength.

$$R ( {λ}_{ex} ( i ) , {λ}_{em} ( k ) ) = D {\sum }_{j = 1}^{{N}_{c}} {C}_{j} {T}_{j} ( {λ}_{ex} ( i ) , {λ}_{em} ( k ) )$$

The system of equations formed by all pairs is then solved for the concentration of each chemical specie.

The light source can be any of the following, including but not limited to a diode laser, light-emitting diode (LED), metal halide lamp, gas arc lamp using xenon, mercury, or a halogen and the like. When the light source 12 is a laser, the laser can be a scanning laser for photon confocal imaging, a scanning laser for two-photon imaging, and the like.

In one embodiment, a measurement is made at one spatial location that corresponds to where the probe is placed in the body. Multiple measurements can also be made over a small region/patch of the tissue site. These measurements can be both parallel (along the organ or mucosal surface) and perpendicular (into the tissue). In one embodiment, when the light source 12 is a scanning laser, two and three dimensional spatial measurements can be taken at surfaces parallel and perpendicular to a tissue site surface. Multiple measurements over a region of the tissue site permit a calculation of the tissue oxygenation gradient, which may be clinically useful information.

By way of illustration, and without limitation, suitable methods for making multiple two-dimensional, along the surface of the organ or mucosa, measurements include but are not limited to: 1) using an imaging endoscope to deliver and collect light; 2) if the light source is a scanning laser, than the excitation beam coming out of the probe end will scan/move across the tissue surface, and the emitted light during each point in the scan reflects the properties of the tissue at that point on the tissue. 3) using a bundle of optical fibers and doing simultaneous or sequential measurements through each of the fibers, and the like.

For measurements perpendicular to the tissue surface (going deep into the tissue) suitable methods include but are not limited to using: 1) confocal imaging (with a pinhole near the detector 16 to reject light that isn't coming from the deeper tissue plane of interest); 2) two-photon imaging, focusing light that has a wavelength twice that of the desired excitation wavelength at the deeper tissue plane; excitation will only become effective close to the focus which is deep in the tissue; 3) using a graded-index (GRIN) lens that provides for a focusing of light coming out of a fiber or endoscope, and also allows more efficiently collect light emitted from the tissue, and the like.

The probe 14 is a light delivery device that is coupled to the light source 12 and focuses the light from the light source 12 into the end of the probe 14. Suitable probes 14 include but are not limited to optical fibers that can be directly or indirectly coupled to the light source, a liquid-light guide, a catheter based probe, an endoscope and the like. The probe 14 can be implantable. Implantable probes 14 are different than the catheter based probes 14 that are inserted into hollow viscous organs, or blood vessels. Suitable implantable probes 14 include but are not limited to, cochlear implants, pace makers, nerve stimulators, deep brain stimulators, and the like. The probe 14 can be a partially implantable probe. Suitable partially implantable probes 14 include but not limited to, diabetic pumps, drainage tubes, implantable feeding tubes such as tubes connecting the intestines to an external port on the abdomen, and the like. The implantable probe 14 can have wireless control for calibration, changing mode of operation, real-time data output, data storage, data-retrieval, battery recharging, and the like. The implantable probe 14 need not need be wireless. In various embodiments, wires or tubes going into the body can be utilized and coupled to the probe 14 that can be surgically removed or simply pulled out at a time after the probe 14 is removed.

The probe 14 can be implanted in a variety of sites that include but are not limited to, the mucosal surface of certain organs such as the gastrointestinal tract and bladder, the parenchyma of other organs such as the kidneys, liver, lungs, heart, and the like. This can be especially useful during transplant surgeries where it is critical to monitor tissue oxygenation after you close incision.

In one embodiment, the system 10, light source 12, detector 16, processor 18 and the like, and not just the probe 14, is contained in a form that can be swallowed. Probe 14 can be a pill version that can be swallowed. In one specific embodiment, the probe 14 provides intermittent or continuous measurements while passing through the GI tract. The pill may also be retrieved after it is passed, allowing for subsequent retrieval of stored measurement data.

In various embodiments, the system calculates the absolute or relative concentrations of one or more molecules selected from the group: elastin, collagen, flavin adenine dinucleotide (FADH₂ and FAD²⁺, nicotinamide adenine dinucleotide (NAD(P)H and NAD(P)⁺), phenylalanine, pyridoxal 5′ phosphate, tryptophan, tyrosine and the like.

There are many clinical scenarios where end-organ perfusion with oxygenated blood can become compromised. In these situations the organ tissue becomes partially or fully deprived of oxygenation. This hypoxic or anoxic state leads to rapid changes in cellular metabolism and respiration. For example, the ability to produce ATP through an aerobic pathway decreases. Commensurate with this process are changes in the concentrations of the key molecules involved in these metabolic pathways. Many of these key metabolic molecules are endogenous fluorphores such as the reduced and oxidized forms of NAD(P) and FAD. Moreover, each of these molecules also has a unique fluorescence excitation and emission spectrum. By using the spectroscopic methods of the present invention, the relative or absolute concentration of one or more of these molecules can be measured. From these values the end organ oxygenation state can be inferred.

### EXAMPLE 1

There are clinical scenarios where tissue can become deprived of oxygen, i.e., hypoxic or anoxic, include but are not limited to, embolic or thrombotic blood vessel stenois or occlusion as in but not limited to, 1) myocardial ischemia, infaractiori, and stroke of the brain, 2) organ transplantation, 3) shock of all types (septic, cardiogenic, hypovolemic) 4) or any other type of organ failure. These situations often arise in the acute care setting such as in an ICU or surgical suite. In all of these situations where there can be a decrease in tissue oxygen perfusion there will be also be changes in the redox state of NAD(P), FAD, and other fluorophores that can be measured. Initially, NAD(P) and FAD shift toward their reduced forms but eventually can switch towards the oxidized state as the tissue dies. The absolute or relative measurements of these fluorphores is then be used to estimate the oxygen perfusion state of the tissue.

### EXAMPLE 2

In the case of shock of any kind, but especially septic, there is often multiple organ failure occurring secondary to decreased perfusion of the end-organs with oxygenated blood. In this scenario, the probe 14 is inserted into a hollow organ, such as the bladder, stomach or rectum, so the end-organ tissue perfusion status is monitored.

In the case where monitoring from the rectum is desired, the probe will be sufficiently small (ideally <1 cm in diameter) so as to easily pass through the anal sphincter. The probe 14 may have light gathering and delivery features such as a GRIN lens attached to its distal end. The probe 14 may also have mechanical and/or chemical adhesive features that promote its attachment and stable interface with intestinal mucosa. For example, the probe may use vacuum suction to attach to the intestinal mucosa. Alternatively, it may bend so as to wedge or lodge itself near the intestinal mucosa. The probe 14 is also be attached to a light conduit such as a bundle of one or more optical fibers, for the purpose of transmitting light from the light source to the probe and in turn transmitting light collected by the probe to the detector.

In operation, the clinician first places the patient in a position amenable for insertion, such as the lateral decubitus. The clinician then inserts the probe 14 through the anal sphincter and advances the probe to a length consistent with desired site of monitoring. In the case of the rectum, this is somewhere between 0-15 cm.

The clinician then activates the system 10 to measure the end-organ oxygen perfusion in the rectum. The system 10 takes its measurement by irradiating the intestinal mucosal surface with light at 380 nm and the emission response is measured at 410 nm and 470 nm. The signal at 410 nm represents mostly background signal that does not change with acute ischemia, while the signal at 470 nm reflects the amount of reduced NAD(P). The intensity of the emitted light is measured separately at 410 nm and 470 nm. The relative or absolute concentration of the reduced NAD(P) is then calculated by dividing the measured intensity at 470 nm by the measured intensity at 410 nm and then multiplying it by a calibration factor that depends on the known excitation and emission properties of the reduced NAD(P). From this calculation of the concentration of the reduced NAD(P), estimate is made of the in vivo cellular energy production status and state of end-organ tissue oxygenation. This estimate is based either upon known relations between the NAD(P)H concentration and in vivo cellular energy production status and state of end-organ tissue oxygenation or upon an experimentally derived calibration. This information is then display and can be used by the clinician to manage patient care accordingly.

### EXAMPLE 3

In the case of solid organ transplantation, maintaining sufficient end-organ oxygen perfusion to the transplanted organ is critical to the survival of the organ. Examples of relevant organ transplantations where monitoring of end-organ oxygen perfusion is beneficial, include but are not limited to the kidney, liver, heart, lung, intestines, limbs, fingers, cornea, and skin.

In the specific case of a kidney transplantation the probe 14 is small, ideally <1 cm, so as to not interfere with transplantation surgery or take up significant volume in the abdominal cavity. The probe 14 attaches to the outer surface of the kidney either through a mechanical means such as a vacuum suction or hooks, or by a chemical adhesive. This attachment is easily reversible and the probe 14 can be removed with minimal to no additional surgery after monitoring is no longer needed. The probe 14 is also attached to a light conduit, such as a bundle of one or more optical fibers, for the purpose of transmitting light from the light source to the probe 14 and in turn transmitting light collected by the probe. 14 to the detector 16.

In operation the clinician attaches the probe 14 to the organ either during the transplantation surgery or immediately after implantation. The clinician activates the system 10 to measure the end-organ oxygen perfusion of the organ. The system 10 determines the state of end-organ oxygen perfusion using a method similar to that described in the case of septic shock above. The probe 14 is left in place after the surgery is completed so measurements of end-organ oxygen perfusion continue in the post-operative period. The probe 14 remains connected to the rest of the system 10 via the light conduit that would pass through a small opening in the abdominal cavity. When monitoring is no longer desired, the probe 14 is removed by retracting it via the light conduit.

### EXAMPLE 4

In many clinical scenarios, including the case of shock mentioned above, it is more desirable to monitor end-organ oxygen perfusion using the bladder as opposed to a site in the GI tract or other place. In this case the probe 14 is sufficiently small enough to pass through the urethra, ideally <5 mm. The probe 14 also has a mechanism to keep it anchored in the bladder. This might be an inflatable balloon near the probe 14 tip similar to that used in a Foley catheter. The probe 14 and light conduit can be integrated into a Foley catheter system for simultaneous use. The probe 14 tip is constructed in a manner such that when the anchoring system is deployed, the tip is placed in contact with the bladder mucosal surface. For example, the probe 14 can be attached to the inflatable balloon in such a way that when it inflates the probe 14 tip is pressed into the bladder mucosal surface. The probe 14 is attached to a light conduit, such as a bundle of one or more optical fibers, for the purpose of transmitting light from the light source 12 to the probe 14 and in turn transmitting light collected by the probe 14 to the detector 16.

In operation, the clinician lubricates the probe 14, inserts the probe 14 through the urethral meatus and advances it until the probe 14 enters the bladder. This distance is approximately 5 cm in women and 15 cm in men. The clinician activates the anchoring mechanism, which may be the inflation of a balloon near the tip of the probe 14. The clinician activates the system 10 to measure the end-organ oxygen perfusion in the bladder. The system 10 determines the state of end-organ oxygen perfusion using a method similar to that described in the case of septic shock above. When monitoring is no longer desired, the probe 14 is removed by deflating the balloon and is retracted via the light conduit.

In one embodiment, the processor 18 take ratios of the measurements at different excitation and emission wavelength pairs to cancel out an effect of at least one of the following: distortions and variation caused by probe placement, optical and instrument distortions, and background signals. In one embodiment the resources 16 use measurements taken of at least one of selected excitation and emission wavelengths where selected species have near identical absorption or emission spectra to estimate an absolute or relative concentration of the sum of the selected species. In another embodiment, the resources 18 use measurements made at multiple spatial locations of the tissue site to determine spatial gradients of at least one of, chemical species, which is then used to determine spatial gradients in in vivo cellular energy production status and state of end-organ tissue oxygenation.

These measurements can be used to determine quantities at the tissue site of at least one of, a percent of reduced NAD(P), a percent of oxidized NAD(P), a NAD(P)+/NAD(P)H ratio, a percent of reduced FAD, a percent of oxidized FAD, a FAD+/FADH₂ ratio, a percent of ischemia, a percent of perfusion, a percent of a perfusion deficit, a tissue state that is aerobic or anerobic, and the like.

The system 10 can include a control module 20 to allow a user to change output displays or view data in a graphical form. The system can also include a GRIN lens, an excitation filter 22, light coupler such as a dichoric mirror 24, mirror 26, emission filter 28 and the like.

In one embodiment, the probe 14 is coupled to an endoscope. In this embodiment, the endoscope functions as both the light conduit and the probe. In another embodiment, the probe 14 is coupled with a pulse oximetry system 30 to make measurements of tissue oxygenation verses blood oxygenation at the same tissue site.

The system 10 can include a mechanical device to anchor the probe 14 to tissue. A vacuum source, hooks, inflatable balloons, expandable cages, and non-toxic chemical adhesives can all be used to anchor the probe 14.

While the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention.

What is claimed is:

1. A method of measuring in vivo of an endogenous fluorophore in a tissue site, comprising:
selecting a known excitation wavelength of the endogenous flurophore within a range of wavelengths at which the endogenous flurophore undergoes fluorescence;
irradiating the tissue site with irradiated light having at least the selected excitation wavelength within the range of wavelengths;
detecting a fluorescence emission of the tissue site resulting from the irradiation thereof;
determining a relative or absolute concentration of the endogenous fluorophore by multiplying it by a calibration factor that depends one at least one of, a known excitation and emission property of the endogenous fluorophore, an intensity of the irradiated light, optical properties of an excitation probe, and specific properties of the tissue;
using the relative or absolute concentration of the endogenous fluorphore to estimate at least one of a, in vivo cellular energy production status or state of end-organ tissue oxygenation.

2. The method of claim 1, wherein the determining is in response to at least one of, known relations between the concentration of the fluorophore and the in vivo cellular energy production status, state of end-organ tissue oxygenation, or upon an experimentally derived calibration.

3. The method of claim 2, further comprising:
detecting reflected and scattered components of the excitation wavelengths incident upon the tissue site.

4. The method of claim 3, wherein absolute or relative concentrations are measured of at least one of the molecules selected from the group elastin, collagen, flavin adenine dinucleotide (FADH2 and FAD2+, nicotinamide adenine dinucleotide (NAD(P)H and NAD(P)+), phenylalanine, pyridoxal 5′ phosphate, tryptophan, and tyrosine.

5. The method of claim 4, wherein fluorescence detections are taken at multiple excitation and multiple emission wavelengths.

6. The method of claim 5, further comprising:
using the fluorescence detections to determine an absolute or relative concentration of one or more chemical species.

7. The method of claim 6, further comprising:
taking ratios of the fluorescence detections at different excitation and emission wavelength pairs to cancel out one or more of the following: background signals that do not vary with wavelength, fluorescent signals from endogenous fluorphores that do not vary with changes in in vivo cellular energy production status and state of end-organ tissue oxygenation, reflected or scattered excitation light, instrumental variation in the optics or other components, and variation in probe placement.

8. The method of claim 7, wherein the fluorescence detections are taken of at least one of selected excitation and emission wavelengths where selected species have near identical absorption or emission spectra to estimate an absolute or relative concentration of the sum of the selected species.

9. The method of claim 8, further comprising:
using fluorescence detections made at multiple spatial locations of the tissue site to determine gradients of at least one of, chemical species, in vivo cellular energy production status, and state of end-organ tissue oxygenation.

10. The method of claim 9, wherein the fluorescence detections are used to determine quantities at the tissue site of at least one of, a percent of reduced NAD(P), a percent of oxidized NAD(P), a NAD(P)+/NAD(P)H ratio, a percent of reduced FAD, a percent of oxidized FAD, a FAD+/FADH2 ratio, a percent of ischemia, a percent of perfusion, a percent of a perfusion deficit, in vivo cellular energy production status, and state of end-organ tissue oxygenation.

11. The method of claim 10, further comprising:
using a control module to allow a user to change output displays or view data in a graphical form.

12. The method of claim 11, further comprising:
using the fluorescence detection to determine at the tissue site an indication of at least one of, end organ perfusion monitoring in an acute care situation, a direct Intra-operative fluorescence detection of tissue oxygenation in transplanted organs, a direct Intra-operative fluorescence detection of tissue oxygenation in surgery, and an evaluation of peripheral vascular disease.

13. The method of claim 12, wherein the tissue site is irradiated with a probe.

14. The method of claim 13, wherein the probe is an implantable apparatus.

15. The method of claim 14, wherein the implantable apparatus is configured to have at least one of, wireless control, data-retrieval, and battery recharging.

16. The method of claim 15, further comprising:
swallowing the probe to irradiate the tissue site.

17. The method of claim 16, wherein the probe provides intermittent or continuous fluorescence detections while passing through the GI tract.

18. The method of claim 17, further comprising:
accessing fluorescence detection data with the probe in real-time with wireless telemetry.

19. The method of claim 18, wherein the probe is recovered from the GI tract with subsequent off-line analysis of fluorescence detection data.

20. The method of claim 19, wherein the tissue site is irradiated with the use of at least one of a, GRIN lens, scanning laser for photon confocal imaging, and a scanning laser for two-photon imaging.

21. The method of claim 20, wherein the fluorescence detections are transvascular, i.e. through a blood vessel wall.

22. The method of claim 21, further comprising:
using a scanning laser to make 2D and 3D spatial fluorescence detections at surfaces parallel and perpendicular to a tissue site surface.

23. The method of claim 22, further comprising:
using a device that provides 2D and 3D spatial fluorescence detections, such as an imaging endoscope, scanning laser, fiber bundle,.GRIN lens, confocal imaging system, and 2 -photon imaging system.

24. The method of claim 23, further comprising:
using a light source selected from the group of LEDS, laser diodes, lasers, metal halide lamps, and gas arc lamps.

25. The method of claim 24, further comprising:
using a detector selected from at least one of a, photodiode, avalanche photodiode, CCD, and PMT.

26. The method of claim 25, further comprising:
using an endoscope with a detector and a light source.

27. The method of claim 26, further comprising:
using pulse oximetry to make fluorescence detections of tissue oxygenation verses blood oxygenation at the tissue site.

28. The method of claim 27, further comprising:
using a mechanical device to anchor the probe to tissue.

29. The method of claim 28, further comprising:
using at least one of, vacuum, hooks, inflatable balloons, chemical adhesives, and expandable cages to anchor the probe.

30. The method of claim 29, wherein oxygenation of in vivo tissue is monitored by measurement of biochemical processes intrinsic to cellular respiration.

31. The method of claim 30, wherein multivariant analysis, using excitation light at 200 nm-420 nm and measuring emission light at 300 nm-800 nm, is used to determine optimal optical wavelengths for detection of the selected one of more fluorphores.

32. A method of measuring in vivo of endogenous fluorophores in a tissue site, comprising:
Irradiating the tissue site at multiple irradiation wavelengths with excitation wavelengths known to excite one or more selected endogenous fluorophores;
receiving a measured emission wavelength response of an emitted light intensity for one or more of the excitation wavelengths;
forming an equation for each measured emission wavelength where the measured emission wavelength is equal to a sum of the responses from the selected endogenous fluorophores; and
forming a system of equations where there is an equation for each combination of an irradiation wavelength and measured emission wavelength.

33. The method of claim 32, further comprising:
solving the system of equations for absolute or relative concentrations of each of the selected endogenous fluorophores. determining a concentration of the selected endogenous fluorophores.

34. The method of claim 33, further comprising:
determining at the tissue site at least one of, in vivo cellular energy production status, and state of end-organ tissue oxygenation.

35. The method of claim 34, wherein the determining at the tissue site at least one of, in vivo cellular energy production status, and state of end-organ tissue oxygenation is based on known relations between the concentrations of the selected endogenous fluorophores and in vivo cellular energy production status and state of end-organ tissue oxygenation or an experimentally derived calibration.

36. The method of claim 35, further comprising:
correcting for at least one of, background endogenous flurophores, instrumental variation, and positioning of a radiation delivery device at the tissue site.

37. The method of claim 36, further comprising:
displaying a result.

38. A method of monitoring energy production status of in vivo tissue, comprising:
measuring emission of first and second groups of endogenous flurophores, the first group having quantity or emitting characteristics that vary with cellular respiration of oxygen, and the second group having quantity or emitting characteristics that vary with energy production status of mammalian cells; and
determining a concentration of one or more of the endogenous fluorophore;
determining a relative or absolute concentration of the endogenous fluorophore by multiplying it by a calibration factor that depends one at least one of, a known excitation and emission property of the endogenous fluorophore, an intensity of the irradiated light, optical properties of an excitation probe, and specific properties of the tissue;
using the relative or absolute concentration of the endogenous fluorophore to estimate at least one of a, in vivo cellular energy production status or state of end-organ tissue oxygenation.

39. The method of claim 38, further comprising:
measuring an electromagnetic signal from intrinsic emission molecules whose quantity or emitting characteristics do not substantially vary in response to a physiological state of energy production status or oxygenation.

40. The method of claim 39, wherein a signal of target molecules and background molecules is separated from endogenous fluorophores whose quantity or emitting characteristics vary significantly in response to physiological variables other than cellular oxygenation or energy production status.

41. The method of claim 40, wherein electromagnetic signals from endogenous fluorophores are separated by at least one of, passive bandwidth filtering, active and or adaptive physical or electronic filtering, and time resolved fluorescent detection to accomplish monitoring of in vivo energy production status or cellular oxygenation.

42. The method of claim 41, wherein a monitoring signal includes emissions of one or more endogenous fluorophores that vary in response to the energy production status or cellular oxygenation of in vivo tissue in combination with the emissions of one or more intrinsic emission molecules whose signal does not vary in response to the energy production status or cellular oxygenation of an in vivo tissue.

43. The method of claim 42, wherein a single band or multiple bands of an electromagnetic signal is isolated by frequency or temporal response monitored to measure in vivo energy production status or cellular respiration.

44. The method of claim 43, further comprising:
determining a ratio from a signal of an endogenous fluorophore or bands of signals from multiple endogenous fluorophores to measure energy production status or cellular respiration of an in vivo tissue.

45. The method of claim 44, wherein energy production status or cellular respiration of an in vivo tissue is measured in at least one of, a single measurement, measured at any frequency, and measured continuously.

46. A system for measuring in vivo at least one of endogenous fluorophores s in a tissue site, comprising:
a light source that produces an excitation wavelength of the endogenous flurophore within a range of wavelengths at which the endogenous flurophore undergoes fluorescence;
a detector for detecting a fluorescence emission of the tissue site resulting from the irradiation thereof; and
a processor configured to analyze the detected emission to determine the presence of the endogenous flurophore in the tissue site.

47. The system of claim 46, wherein the detector detects reflected and scattered components of the excitation wavelengths incident upon the tissue site.

48. The system of claim 47, wherein the system measures absolute or relative concentrations of at least one of at least one molecule selected from the group elastin, collagen, flavin adenine dinucleotide (FADH2 and FAD2+, nicotinamide adenine dinucleotide (NAD(P)H and NAD(P)+), phenylalanine, pyridoxal 5′ phosphate, tryptophan, and tyrosine.

49. The system of claim 48, wherein the system takes measurements at multiple excitation and multiple emission wavelengths are taken.

50. The system of claim 49, wherein the system uses the measurements to determine an absolute or relative concentration of 1 or more chemical species.

51. The system of claim 50, wherein the processor takes ratios of the fluorescence measurements at different excitation and emission wavelength pairs to cancel out one or more of the following: background signals that do not vary with wavelength, fluorescent signals from endogenous fluorphores that do not vary with changes in in vivo cellular energy production status and state of end-organ tissue oxygenation, reflected or scattered excitation light, instrumental variation in the optics or other components, and variation in probe placement.

52. The system of claim 51, wherein the processor uses measurements taken of at least one of selected excitation and emission wavelengths where selected species have near identical absorption or emission spectra to estimate an absolute or relative concentration of the sum of the selected species.

53. The system of claim 52, wherein the processor uses measurements made at multiple spatial locations of the tissue site to determine gradients of at least one of, chemical species, in vivo cellular energy production status, and state of end-organ tissue oxygenation.

54. The system of claim 53, wherein the measurements are used to determine quantities at the tissue site of at least one of, a percent of reduced NAD(P), a percent of oxidized NAD(P), a NAD(P)+/NAD(P)H ratio, a percent of reduced FAD, a percent of oxidized FAD, a FAD+/FADH2 ratio, a percent of ischemia, a percent of perfusion, a percent of a perfusion deficit, a tissue state that is aerobic or anerobic.

55. The system of claim 54, further comprising:
a control module to allow a user to change output displays or view data in a graphical form.

56. The system of claim 55, wherein the tissue site is irradiated with a probe.

57. The system of claim 56, wherein the probe is an implantable apparatus.

58. The system of claim 57, wherein the implantable apparatus is configured to have at least one of, wireless control, data-retrieval, and battery recharging.

59. The system of claim 58, wherein the probe provides intermittent or continuous measurements while passing through the GI tract.

60. The system of claim 59, wherein the probe accesses measurement data with the probe in real-time with wireless telemetry.

61. The system of claim 60, wherein the probe is recovered from the GI tract with subsequent off-line analysis of measurement data.

62. The system of claim 61, further comprising at least one of a GRIN lens, dichroic mirror, excitation filter, emission filter, and a light conduit.

63. The system of claim 62, wherein the light source is selected from an LED, laser diode, laser, metal halide lamp, gas arc lamp, scanning laser for confocal imaging, and a scanning laser for 2-photon imaging.

64. The system of claim 63, wherein the measurements are through a blood vessel wall.

65. The system of claim 64, wherein the light source is a scanning laser and 2D and 3D spatial measurements are at surfaces parallel and perpendicular to a tissue site surface.

66. The system of claim 65, further comprising:
a device to provide 2D and 3D spatial measurements such as an imaging endoscope, scanning laser, fiber bundle, GRIN lens, confocal imaging system, and 2-photon imaging system.

67. The system of claim 66, wherein the light source is selected from the group of LEDS, laser diodes, lasers, and an arc lamp.

68. The system of claim 67, wherein the detector is selected from at least one of a, photodiode, avalanche photodiode, CCD, and PMT.

69. The system of claim 68, further comprising:
an endoscope used with the detector and the light source.

70. The system of claim 69, further comprising:
a pulse oximetry system to make measurements of tissue oxygenation verses blood oxygenation at the tissue site.

71. The system of claim 70, further comprising:
a mechanical device to anchor the probe to tissue.

72. The system of claim 71, further comprising:
at least one of a vacuum source, hooks, inflatable balloons and expandable cages to anchor the probe.

73. The system of claim 72, further comprising:
an delivery device coupled to the probe.

74. The system of claim 73, wherein the delivery device is a catheter configured to be placed through at least one of the, mouth, nasal cavity, rectum and urethrea, intravascularly, and into a body cavity by penetration of the skin surface.

75. The system of claim 74, further comprising:
a device that passes excitation, light from the light source outside the body through a conduit to a location inside the body to be used for the excitation of the endogenous fluorophores.

76. The system of claim 75, wherein the device that passes excitation light is selected from at least one of, a fiber optic element and a device that contains one or more liquid light guide elements.

77. The system of claim 76, further comprising:
a device that collects light emitted by the endogenous fluorophores and acts as a conduit for that light to be measured.

78. The system of claim 77, wherein the device that collects light is selected from at least one of, a fiber optic element that transmit the light from an in vivo region to the detector and a liquid light guide elements to transmit the light from the in vivo region to a detection apparatus outside the body for this purpose.

79. The system of claim 78, further comprising:
a device that acts as a conduit to pass excitation light from the light source outside a body and collects light emitted by the endogenous fluorophores.

80. The system of claim 79, wherein the device that acts as a conduit is selected from at least one of, a fiber optic element and a liquid light guide element.

81. The system of claim 80, wherein the light source is insertable into a body of the tissue site.

82. The system of claim 81, wherein the detector is insertable into a body of the tissue site.

83. The system of claim 82, wherein the light source and the detector are combined in a single device that is insertable into a body of the tissue site.

84. The system of claim 83, further comprising:
a disposable light delivery deliver coupled to the light source and insertable into a body of the tissue site.

85. The system of claim 84, further comprising:
a device that is segmented into disposable and reusable components for the purpose of exciting or collecting light emission of the endogenous fluorophores.

86. The system of claim 85, further comprising:
a device that excites or collects emission of endogenous fluorophores with a separate lumen suitable for the passing or removing fluids.

87. The system of claim 86, wherein the device that excites or collects emission is inserted through at least one of a, nose, mouth, urethra and vasculature.

88. The system of claim 87, further comprising:
a device that has at least one of a, unidirectional, multidirectional and omni-directional optical tip inserted into a body of the tissue site.

89. The system of claim 88, further comprising:
a device external from a body of the tissue site configured to be coupled to a probe inserted into the body.

90. The system of claim 89, wherein the light source includes optical and electronic elements.

91. The system of claim 90, wherein the detector includes optical and electronic elements.

92. The system of claim 91, further comprising:
a device external from a body of the insertable device that provides processing, filtering, and reporting of signals acquired from emission of the endogenous fluorophores.

93. The system of claim 92, wherein the light source is a scanning laser selected from a, confocal scanning laser and a two photon scanning laser.

94. The system of claim 93, wherein an electrically-acutated movablemicro-mirror is used to provide scanning.
